(19) United States
(12) Patent Application Publication
(10) Pub. No.: US 2025/0176927 A1
(43) Pub. Date: Jun. 5, 2025

(54) MODULE CARRIER FOR A CT DETECTOR MODULE, CT DETECTOR MODULE AND CT DEVICE

(71) Applicant: Siemens Healthineers AG, Forchheim (DE)

(72) Inventors: Lukas RUHMANN, Gremsdorf (DE); Michael TEUBER, Forchheim (DE); Oleksandr MAKSYMENKO, Zwickau (DE); Gottfried TSCHOEPA, Baiersdorf (DE); Stefan WOELFEL, Dormitz (DE)

(73) Assignee: Siemens Healthineers AG, Forchheim (DE)

(21) Appl. No.: 18/961,548

(22) Filed: Nov. 27, 2024

(30) Foreign Application Priority Data
Nov. 30, 2023 (DE) 10 2023 211 985.4

(51) Int. Cl. A61B 6/00; A61B 6/03

(52) U.S. Cl. CPC A61B 6/4488; A61B 6/035; A61B 6/4411

(57) ABSTRACT

One or more example embodiments relates to a module carrier for a CT detector module. The module carrier includes a one-piece tunnel-shaped hollow body, the hollow body including an elongated hollow space and at least two openings, two openings of the at least two openings are opposite one another along a longitudinal direction of the hollow body, the hollow body configured to, be arranged on an X-ray converter element on an outer side of the hollow body, receive at least one cooling element in the hollow space via at least one of the at least two openings, and enable a fluid to flow through the hollow body along the longitudinal direction of the hollow body, wherein the hollow body has a heat transfer section configured to provide a heat-conducting contact between the at least one cooling element and the X-ray converter element.

### CROSS-REFERENCE TO RELATED APPLICATION(S)

The present application claims priority under 35 U.S.C. § 119 to German Patent Application No. 10 2023 211 985.4, filed Nov. 30, 2023, the entire contents of which is incorporated herein by reference.

### FIELD

One or more example embodiments relates to a module carrier for a CT detector module, a CT detector module and a CT device.

### RELATED ART

Modern computed tomography devices (CT devices) have a gantry with a rotatable frame on which, among other things, the X-ray source and a detector module for detecting the X-ray radiation are arranged. A CT detector module of this type usually comprises an X-ray converter element, which has an X-ray sensor layer and possibly a layer with A/D (analog-to-digital) converters arranged beneath it. Electronic integration has recently been an important trend in the case of X-ray converter elements. The aim was to reduce the length of the analog path between the analog X-ray sensor layer and the A/D converters, which are usually implemented as application-specific integrated circuits (ASICs). In integrating X-ray converters, the analog X-ray sensor layer is formed by a suitable sensor layer, for example a scintillator, in combination with a photodiode, whereas in counting X-ray converters a direct-conversion semiconductor sensor is used. An A/D converter designed as an ASIC then generates the digital output signal. In both cases, integrating the ASICs into a compact structure, in particular a stack structure, together with the analog X-ray sensor layer, brings a significant heat source closer to the X-ray sensor elements of the X-ray sensor layer itself.

Since the X-ray sensor elements are very sensitive to temperature fluctuations, heat management is a crucial task in the development of a modern CT detector module. The particular challenges of heat management are to keep the operating temperature of the CT detector module stable, to avoid temperature gradients between neighboring X-ray converter elements and also to reduce the temperature gradients within each X-ray converter element. These challenges are even more important for counting X-ray converters because the directly converting semiconductor sensors are additional heat sources and at the same time their sensor performance is very sensitive to thermal changes.

Currently, heat management is ensured by creating a thermal interface between the X-ray converter element and the metal frame of the CT detector module using a heat-conducting adhesive or heat-conducting paste.

### SUMMARY

However, this does not allow a defined amount of heat to be transported because the quality of the thermal interface depends on various factors that cannot be easily influenced, in particular the thickness of the gap between the X-ray converter and the metal frame, as well as the exact distribution of the heat-conducting paste.

Due to the high data volumes of modern CT (counting) detectors, there is an increasing demand for efficient cooling of the modules to keep the sensors made of semiconductor material within their ideal operating temperature range owing to the high power of the ASICs. In addition to the absolute temperature, the temperature distribution over the sensor area also plays a crucial role. As a rule, the sensor elements closer to the cooling air inlet openings have a lower temperature because a large proportion of the incoming cooling air is still present here and the cooling fins are exposed to frontal airflow. As a result, additional air is displaced into the rear space of the detector, and the sensors in the area facing away from the inlet opening thus receive less cooling air, which is also heated by the other sensor boards.

One or more example embodiments provides more efficient heat dissipation for a CT detector module.

Independent of the grammatical term usage, individuals with male, female or other gender identities are included within the term.

### BRIEF DESCRIPTION OF THE DRAWINGS

Exemplary embodiments of the invention are illustrated in the drawings and are further described below. The identical reference characters are used for identical features in the different figures. In the drawings:

FIGS. 1 and 2 show schematic representations of different advantageous embodiments of a module carrier,

FIGS. 3 to 5 show schematic representations of different advantageous embodiments of a CT detector module, and

FIG. 6 shows a schematic representation of an advantageous embodiment of a CT device.

### DETAILED DESCRIPTION

One or more example embodiments relates in a first aspect to a module carrier for a CT detector module. In this case, the module carrier has a one-piece and tunnel-shaped hollow body. The hollow body has an elongated hollow space and at least two openings. In this case, two openings of the at least two openings are arranged opposite one another along a longitudinal direction of extent of the hollow body. The hollow body is designed to be arranged on its outer side on an X-ray converter element. In addition, the hollow body is designed to receive at least one cooling element by insertion by way of at least one of the at least two openings in its hollow space. Furthermore, the hollow body is designed to enable a fluid to flow through along its longitudinal direction of extent. In this case, the hollow body has a heat transfer section which is designed to provide a heat-conducting contact between the at least one cooling element and the X-ray converter element.

The one-piece, in particular single-piece, production of the hollow body can advantageously reduce the constructive effort involved in producing the hollow body. For example, it can save on materials and fastening means, as well as time-consuming processing steps. In addition, when designed as one piece, the hollow body can be advantageously completely formed from an in particular metal material, in particular a substrate. This allows the hollow body to exhibit homogeneous thermal conductivity. For example, the hollow body can be made from an extruded profile with subsequent mechanical processing.

The hollow body can be tunnel-shaped, in particular tubular and/or hose-shaped and/or cylindrical and/or cuboid. In this case, the elongated hollow space of the hollow body can be tubular and/or hose-shaped and/or cylindrical and/or cuboid. The hollow space can be surrounded by the, in particular metal, material along a peripheral surface around the longitudinal direction of extent of the hollow body, in particular delimited by the material. In particular, the hollow space can be surrounded by the material along directions different from the longitudinal direction of extent of the hollow body. Advantageously, the hollow space can run from one side of the hollow body along the longitudinal direction of extent continuously to an opposite side of the hollow body, in particular in a straight or curved line. Moreover, the hollow body can have respectively an opening along its direction of longitudinal extent at each of its two ends. The two openings can be designed geometrically identical or different, by way of example with respect to their area and/or contour.

Advantageously, the hollow space can run in a straight line, in particular essentially without curvature, along the longitudinal direction of extent of the hollow body. In this case, the walls of the hollow body, for example a peripheral surface of the hollow body, can run essentially parallel to the longitudinal direction of extent of the hollow body. This can enable a grid-like arrangement of multiple CT detector elements, in particular module carriers, adjacent to one another. In addition, it can enable improved flow through the hollow body by the fluid.

The hollow body, in particular the elongated hollow space, is designed to enable the fluid, for example a liquid, in particular water, and/or a gas, in particular air, to flow through along its longitudinal direction of extent, in particular from one side of the hollow body along the longitudinal direction of extent to the opposite side of the hollow body. In particular, the hollow body, in particular the hollow space, can be designed to guide the fluid along its longitudinal direction of extent, in particular to transport it.

Furthermore, the hollow body can be designed to receive at least one cooling element, in particular multiple cooling elements, by insertion by way of at least one of the at least two openings in its hollow space. In particular, the hollow body can be designed to receive the at least one cooling element, in particular the multiple cooling elements, by insertion by way of at least one of the two opposite-lying openings or a further opening in its hollow space. Advantageously, the at least one cooling element can be introduced, in particular pushed, into the hollow space of the hollow body by way of the at least one opening. In this case, the hollow body can be designed to receive, in particular hold, the at least one cooling element in a target positioning. The target positioning can describe a spatial relative position and/or relative orientation and/or relative pose of the at least one cooling element with respect to the hollow body. For the positioning of the at least one cooling element in its target position, the hollow body can have on its inner side, for example, a positioning element, in particular a raised area and/or recess for guiding and/or positioning and/or fixing the at least one cooling element.

The at least one cooling element can have a heat contact surface. Furthermore, the cooling element can be designed to enable fluid to flow through in its target position along the longitudinal direction of extent of the hollow body.

The hollow body is designed to be arranged on the X-ray converter element on its outer side, in particular on a side, in particular surface, facing away from the hollow space. The X-ray converter element can have an X-ray detector layer on its upper side. Furthermore, the X-ray detector layer can be designed to detect X-ray radiation emitted by the X-ray source. The X-ray detector layer can comprise a direct-conversion (semiconductor) X-ray sensor layer, for example comprising CdTe, CdZnTe, CdTeSe, CdZnTeSe, CdMnTe, GaAs, Si or Ge as semiconductor material. The X-ray detector layer can also comprise an X-ray sensor layer that is designed to convert X-ray radiation into light and optically coupled photodiodes, in particular one or multiple photodiode arrays. Scintillator material, for example GOS (Gd2O2S), CsJ, YGO or LuTAG, is often used as the material. The X-ray detector layer can also comprise a layer with analog-to-digital converters, onto which the X-ray sensor layer is applied, wherein the A/D converter layer can be realized in one or multiple ASICs. The X-ray detector layer can be applied to a base plate, also referred to as a substrate, for example a printed circuit board or a ceramic or glass substrate, which then forms an underside of the X-ray converter element. The X-ray converter element can have a heat contact surface, in particular a metal heat contact surface, on its underside. The heat contact surface can cover part or all of the underside of the X-ray converter element. The heat contact surface can be connected to that of the A/D converter layer and/or the X-ray sensor layer by metallized through-holes in a base plate and/or the A/D converter layer of the X-ray detector layer, so that the heat can be conducted from there into the heat contact surface. This is also referred to as thermal via technology. The metallized through-holes can, for example, be contact holes in the base plate.

The hollow body has a heat transfer section, in particular multiple heat transfer sections, which is designed to provide a heat-conducting, in particular thermal, contact between the at least one cooling element, in particular the heat contact surface of the at least one cooling element, and the X-ray converter element, in particular the heat contact surface of the X-ray converter element. Advantageously, the heat transfer section can be arranged at the target positioning of the at least one cooling element. The heat transfer section can be designed to transfer heat between the X-ray converter element, in particular the heat contact surface of the X-ray converter element, and the at least one cooling element, in particular the heat contact surface of the at least one cooling element.

The heat transfer section can provide direct or indirect heat-conducting contact between the X-ray converter element and the at least one cooling element. In the case of direct heat-conducting contact, the two heat contact surfaces of the X-ray converter element and of the at least one cooling element can directly contact, in particular touch, one another by virtue of a corresponding recess, in particular opening, of the hollow body. In particular, the two heat contact surfaces of the X-ray converter element and of the at least one cooling element can be pressed against one another or adhered to one another. In the case of indirect heat-conducting contact, the heat transfer through the material of the hollow body can be conveyed at the heat transfer section between the heat contact surfaces of the X-ray converter element and of the at least one cooling element, for example by virtue of corresponding heat contact surfaces on an outer side and inner side of the hollow body. In particular, the heat contact surface on the inner side of the hollow body and the heat contact surface of the at least one cooling element can be pressed against one another or adhered together. Furthermore, the heat contact surface on the outer side of the hollow body and the heat contact surface of the X-ray converter element can be pressed against one another or adhered together.

The heat-conducting contact can be further improved by, for example, heat-conducting pastes or heat-conducting pads or adhesives or soldering materials. The heat contact surfaces can, for example, have a metal coating. The metal coating can, for example, be formed by coating with a metal that is a good heat conductor, for example gold, silver or copper. The metal coating can be a so-called “metallized thermal pad”, a metallized heat-conducting pad.

An embodiment can advantageously enable more efficient heat dissipation for a CT detector module.

In a further advantageous embodiment of the module carrier, the hollow body can have a further opening, which is arranged away from its longitudinal direction of extent. In this case, the hollow body can be designed to receive the at least one cooling element by insertion by way of the further opening in its hollow space.

In particular, the hollow body can be designed to receive multiple cooling elements by insertion by way of the further opening in its hollow space. Advantageously, the at least one cooling element can be inserted, in particular pushed, into the hollow space of the hollow body by way of the further opening. The further opening can be arranged on an outer side, in particular a peripheral surface, of the hollow body away from its longitudinal direction of extent. In particular, the further opening can be arranged on the outer side of the hollow body opposite the X-ray converter element.

The embodiment can enable simple and positionally precise insertion of the at least one cooling element into the hollow space of the hollow body. In addition, the embodiment can advantageously enable that the two opposite-lying openings can be designed for an optimal through-flow of the fluid. In particular, the two opposite-lying openings do not necessarily have to be designed for inserting the at least one cooling element into the hollow space.

In a further advantageous embodiment of the module carrier, the module carrier can have a cover element. In an operating state of the module carrier, the at least one cooling element can be arranged in the hollow space and the cover element can be fastened to the hollow body. In addition, the cover element can seal the further opening at least partially, in particular completely.

The cover element can be designed for example as a cover plate, in particular made of a metal material. In particular, the cover plate can be formed from the same metal material as the hollow body. In the operating state, the cover element can be fastened to the hollow body, in particular the further opening, via a fastening means. The fastening means can be or at least include, for example, a mechanical fastening means, in particular a screw, a pin, a bolt, a rivet and/or a spring, and/or a chemical fastening means, in particular an adhesive. Advantageously, the fastening means can be designed to fasten the cover element to the hollow body, in particular in a fixed manner. The cover element can be advantageously designed to seal the further opening in a fluid-tight manner. Moreover, the cover element can be designed to hold the at least one cooling element in its target positioning in the hollow space of the hollow body.

The embodiment can enable an improved flow of the fluid through the hollow space. In addition, the cover element can improve a shielding against primary radiation in order to protect further electronic components that are arranged behind it. Furthermore, the cover element can advantageously contribute to a further reinforcing of the hollow body.

In a further advantageous embodiment of the module carrier, the hollow body can be designed to receive multiple cooling elements by way of at least one of the at least two openings in its hollow space. Moreover, the multiple cooling elements can be arranged along the longitudinal direction of extent of the hollow body in a row in the hollow space.

In this case, respectively one of the cooling elements along the longitudinal direction of extent can adjoin at least one further cooling element, in particular it can rest against the at least one further cooling element or be arranged at a distance from the at least one further cooling element. The multiple cooling elements can each have at least one hollow space which runs along the longitudinal direction of extent of the hollow body when the cooling elements are arranged in a row in the hollow space. The hollow spaces of the cooling elements can each be designed tunnel-shaped, in particular tubular and/or hose-shaped and/or cylindrical and/or cuboid. In this case, the hollow spaces can be surrounded by the, in particular metal, material of the cooling elements, in particular delimited by the metal material. Advantageously, the hollow spaces of the cooling elements can each run from one side of the cooling element along the longitudinal direction of extent continuously to an opposite side of the cooling element, in particular in a straight line or curved. The hollow spaces can each have an opening on both sides of the cooling elements. Furthermore, the cooling elements can be arranged in a row in the hollow body in such a way that the openings of the hollow spaces of respectively adjacent cooling elements overlap at least partially, in particular completely.

It is possible by virtue of the multiple cooling elements for the embodiment to enable efficient heat dissipation along the longitudinal direction of extent of the hollow body, in particular adapted to the waste heat of the X-ray converter element along the longitudinal direction of extent.

In a further advantageous embodiment of the module carrier, the hollow body can have respectively a heat transfer section for each of the cooling elements. In this case, the cooling elements in the row-shaped arrangement can have a different length along the longitudinal direction of extent of the hollow body. In addition, a length of the heat transfer sections along the longitudinal direction of extent of the hollow body can be adapted to the length of the cooling elements.

The multiple heat transfer sections can be arranged on a common side of the hollow body or at least in part on different sides of the hollow body. Moreover, the cooling elements when arranged in a row can have a different length, in particular spatial extent, along the longitudinal direction of extent of the hollow body. By way of example, the cooling elements can have a reducing length when arranged in a row along the longitudinal direction of extent of the hollow body. In this case, the length of the heat transfer sections, in particular a length of the openings or a length of the heat contact surfaces of the hollow body, along the longitudinal direction of extent of the hollow body can be adapted to the length of the cooling elements, in particular to a respective length of the heat contact surfaces of the cooling elements.

The embodiment can enable a homogeneous heat dissipation along the longitudinal direction of extent of the hollow body by virtue of the multiple cooling elements.

In a further advantageous embodiment of the module carrier, the heat transfer section can have respectively an opening for each of the cooling elements to be received. In this case, the at least one cooling element can have a direct heat-conducting contact with the X-ray converter element in an operating state of the module carrier when arranged in the hollow space.

The heat transfer section can have a corresponding opening, in particular a recess, on the hollow body, in particular the material of the hollow body, for each of the cooling elements to be received. The at least one opening can run continuously from the inner side, in particular the hollow space, to the outer side of the hollow body. Advantageously, the at least one opening can be arranged on the hollow body in such a way that, in the operating state of the module carrier, direct heat-conducting contact between the heat contact surfaces of the X-ray converter element and the at least one cooling element is provided by virtue of the opening. In particular, the heat transfer section can have multiple openings, which are arranged on the hollow body in such a way that, in the operating state of the module carrier, direct heat-conducting contact between the heat contact surfaces of the X-ray converter element and the several cooling elements is provided by virtue of the respective opening.

The embodiment can enable a particularly efficient, in particular direct, heat transfer between the X-ray detector element and the at least one cooling element.

In a further advantageous embodiment of the module carrier, the heat transfer section can have respectively a heat contact surface on the inner side of the hollow body for each of the cooling elements to be received and on the outer side at least one further heat contact surface. In this case, the heat transfer section can be designed to transfer heat between the heat contact surfaces of the inner side and outer side of the hollow body. In an operating state of the module carrier, the at least one cooling element, when arranged in the hollow space, can have a direct heat-conducting contact with the respective heat contact surface on the inner side. In addition, in the operating state, the X-ray converter element, when arranged on the outer side of the hollow body, can have a direct heat-conducting contact with the heat contact surface on the outside.

Advantageously, the heat transfer section can have respectively a heat contact surface on the inner side and the outer side of the hollow body for each of the cooling elements to be received. The heat contact surfaces corresponding to each of the cooling elements to be received can be arranged offset or opposite one another on the inner side and outer side of the hollow body. The corresponding heat contact surfaces can be formed identically or differently with regard to their material composition and/or geometric features, for example a spatial extent, in particular surface, and/or contour and/or shape and/or a pattern.

The heat transfer section can also be designed to transfer heat between the corresponding heat contact surfaces of the inner side and outer side of the hollow body, in particular to conduct and/or transfer heat. The heat transfer can be effected via the, in particular metal, material of the hollow body, which is arranged between the corresponding heat contact surfaces of the inside and outside.

Advantageously, in the operating state of the module carrier, the at least one cooling element, in particular the multiple cooling elements, when arranged in the hollow space, can have a direct heat-conducting contact with the respective heat contact surface on the inner side of the hollow body. Advantageously, the heat contact surface can be arranged on the inside of the hollow body in such a way that, in the operating state of the module carrier, direct heat-conducting contact is provided between the heat contact surface of the at least one cooling element and the inner-lying heat contact surface of the heat transfer section. In particular, the heat transfer section can have several heat contact surfaces, which are arranged on the inner side of the hollow body in such a way that, in the operating state of the module carrier, a direct heat-conducting contact is provided between the heat contact surfaces of the cooling elements and the inner-lying heat contact surfaces of the heat transfer section.

Advantageously, in the operating state of the module carrier, the X-ray converter element, when arranged on the outer side of the hollow body, can have a direct heat-conducting contact with the heat contact surface on the outer side of the hollow body. Advantageously, the heat contact surface can be arranged on the outer side of the hollow body in such a way that, in the operating state of the module carrier, a direct heat-conducting contact is provided between the heat contact surface of the X-ray converter element and the outer-lying heat contact surface of the heat transfer section.

The embodiment can enable heat transfer via the hollow body, in particular the material of the hollow body. As a consequence, an efficient heat dissipation can be enabled.

In a further advantageous embodiment of the module carrier, the hollow body can have at least one first and at least one further section along its longitudinal direction of extent. In this case, the at least one first section can be designed to receive the at least one cooling element. Furthermore, the at least one second section can have a flow-conducting internal structure.

Advantageously, the hollow body can have multiple, in particular spatial, sections, in particular one or multiple first sections and one or multiple further sections along its longitudinal direction of extent. The at least one first and the at least one further section can be arranged directly adjacent to one another or spaced apart from one another. In addition, a further section or multiple further sections can be arranged before or after one or multiple first sections or between two first sections, or vice versa.

The at least one first section can be designed to accommodate the at least one cooling element. In particular, the hollow body can have a first section for each cooling element to be received. The at least one first section can, for example, be designed to hold the at least one cooling element in a form-fitting manner, in particular in its target positioning. For example, the at least one first section can have on the inner side of the hollow body a recess and/or elevation and/or structure which is adapted to a shape, in particular a recess and/or elevation and/or structure, on the outer side of the at least one cooling element to be received.

The at least one further section can have a flow-conducting internal structure, for example a recess and/or elevation. Advantageously, the at least one further section can be designed to adapt a flow of the fluid flowing through the hollow body in the operating state in a defined manner, for example to slow it down and/or accelerate it and/or deflect it and/or mix it and/or swirl it.

In an advantageous embodiment, the hollow body can be designed to receive multiple cooling elements in a row-shaped arrangement. Furthermore, the hollow body can have respectively a first section for each of the cooling elements to be received. In this case, the hollow body can have two further sections, which are arranged before and after a row-shaped arrangement of the multiple first sections.

In a further advantageous embodiment of the module carrier, the hollow body can have a closed profile transverse to its longitudinal direction.

Advantageously, the hollow space of the hollow body can be completely surrounded by the material of the hollow body away from the longitudinal direction of extent. Thus, the hollow body can have a completely closed profile in a cross-section perpendicular to its longitudinal direction of extent and surrounding the hollow space. The profile can be, for example, polygonal or oval in shape

The embodiment can advantageously enable a reduction in flow loss in a rear space when the fluid flows through the hollow space, since the inflowing fluid, for example a cooling air, can be almost completely supplied to the at least one cooling element and used for cooling the X-ray converter element. The closed and more rigid shape of the hollow body allows the required wall thicknesses to be advantageously minimized. This can maximize a cross-section of the hollow body available for the through-flow of the fluid and a surface of the cooling element used for convection. In addition, hollow bodies with a closed profile can be better suited for mechanical post-processing, since less mechanical distortion and/or less mechanical deformation due to rotational forces, in particular torsion, can occur. As a result, the blank hollow bodies can be processed in a modular fashion to form the hollow bodies, and the hollow bodies can be fixed in a detector module carrier with greater precision at a later stage. Advantageously, the hollow body can be made from an extruded profile with subsequent mechanical processing. This form of production is comparable in terms of production costs to forms of production such as aluminum die casting with subsequent mechanical processing, but in contrast to this, it has the advantages described above.

One or more example embodiments relates in a second aspect to a CT detector module, which comprises at least one X-ray converter element, a module carrier according to one or more example embodiments and at least one cooling element. The X-ray converter element has an upper side and an underside. Furthermore, the X-ray converter element has an X-ray detector layer on its upper side and a heat contact surface on its underside. In an operating state of the CT detector module, the at least one cooling element is arranged in the hollow space of the hollow body. Furthermore, in the operating state, the X-ray converter element is arranged on the outer side of the hollow body. Furthermore, the at least one cooling element is in heat-conducting contact with the heat contact surface of the X-ray converter element by way of the heat transfer section in the operating state.

The advantages of the CT detector module essentially correspond to the advantages of the cooling apparatus. Features, advantages or alternative embodiments mentioned herein may also be applied to the other claimed subjects and vice versa.

The X-ray converter element has an X-ray detector layer on its upper side. In an operating state of the CT detector module, this can face the X-ray source. Furthermore, the X-ray detector layer can be designed to detect X-ray radiation emitted by the X-ray source. The X-ray detector layer can comprise a direct-converting (semiconductor) X-ray sensor layer, for example comprising CdTe, CdZnTe, CdTeSe, CdZnTeSe, CdMnTe, GaAs, Si or Ge as semiconductor material. The X-ray detector layer can also comprise an X-ray sensor layer that is designed to convert X-ray radiation into light and optically coupled photodiodes, in particular one or multiple photodiode arrays. Scintillator material, for example GOS (Gd2O2S), CsJ, YGO or LuTAG, is often used as the material. The X-ray detector layer can also comprise a layer with analog-to-digital converters, onto which the X-ray sensor layer is applied, wherein the A/D converter layer can be realized in one or multiple ASICs. The X-ray detector layer can be applied to a base plate, also known as a substrate, for example a printed circuit board or a ceramic or glass substrate, which then forms the underside of the X-ray converter element.

The X-ray converter element has on its underside a heat contact surface, in particular a metal heat contact surface, which is in heat-conducting, in particular thermal, contact with the cooling apparatus in the operating state of the CT detector module. The heat-conducting contact can be further improved by, for example, heat-conducting pastes or heat-conducting pads or adhesives or soldering materials. In the operating state, the multiple cooling elements of the cooling apparatus can be arranged in a row on the underside of the X-ray converter element in such a way that the respective recess and/or elevation of the cooling elements is in heat-conducting contact with the respectively corresponding heat contact surface of the X-ray converter element. For this purpose, the cooling elements can, for example, be arranged in a common module carrier which allows the row-shaped arrangement of the cooling elements and has a recess on the at least one heat contact surface. The at least one heat contact surface can, for example, have a metal coating. The metal coating can, for example, be formed by a coating with a metal which is a good heat conductor, for example gold, silver or copper. The metal coating can be a so-called “metallized thermal pad”, a metallized heat-conducting pad. The at least one heat contact surface can cover a part of or also the entire underside of the X-ray converter element. The heat contact surface can be connected to that of the A/D converter layer and/or the X-ray sensor layer by metallized through-holes in a base plate and/or the A/D converter layer of the X-ray detector layer, so that the heat can be conducted from there into the heat contact surface. This is also referred to as thermal via technology. The metallized through-holes can, for example, be contact holes in the base plate.

The embodiment can enable improved heat dissipation from the X-ray detector element to the at least one cooling element. In particular, by arranging the at least one cooling element in the hollow body, a gap between the cooling element and the hollow body or between the cooling element and the X-ray converter element, in particular the underside of the X-ray converter element, can be significantly reduced, since no tolerance compensation is required. This can further improve the heat dissipation of the X-ray converter element.

In a further advantageous embodiment of the CT detector module, the at least one cooling element in the arrangement in the hollow space of the hollow body can be designed to enable a fluid to flow through along the longitudinal direction of extent of the hollow body. Furthermore, in the operating state of the CT detector module, the fluid can flow along the longitudinal direction of extent of the hollow body through the hollow body and the at least one cooling element arranged therein.

The embodiment can enable improved heat dissipation from the X-ray detector element to the fluid via the at least one cooling element.

In a further advantageous embodiment of the CT detector module, the CT detector module can comprise multiple cooling elements, which in the operating state of the CT detector module are arranged in a row in the hollow space along the longitudinal direction of extent of the hollow body.

In a further advantageous embodiment of the CT detector module, the heat transfer section can have respectively one opening for each of the cooling elements to be received. Moreover, the at least one cooling element in the operating state can be in direct heat-conducting contact with the heat contact surface of the X-ray converter element and can be fastened to the X-converter element via a fastening means.

The fastening means can be or at least comprise, for example, a mechanical fastening means, in particular a screw, a pin, a bolt, a rivet and/or a spring, and/or a chemical fastening means, in particular an adhesive. Advantageously, the fastening means can be designed to fasten the at least one cooling element, in particular the multiple cooling elements, to the X-ray converter element, in particular to hold it in a fixed position. In particular, the fastening means can be designed to fasten the at least one cooling element to the X-ray converter element by way of the at least one opening of the hollow body.

The embodiment can enable a positionally accurate arrangement of the at least one cooling element with respect to the X-ray converter element that is to be cooled.

In a further advantageous embodiment of the CT detector module, the at least one cooling element can be fastened to the hollow body via a further fastening means in the operating state.

The further fastening means can be the same as or different from the fastening means for fastening the cooling element to the X-ray converter element. The further fastening means can, for example, comprise a mechanical fastening means, in particular a screw, a pin, a bolt, a rivet and/or a spring, and/or a chemical fastening means, in particular an adhesive. Advantageously, the further fastening means can be designed to fasten the at least one cooling element, in particular the multiple cooling elements, to the hollow body, in particular to an inner side of the hollow body, in particular to hold it in a fixed position.

In a third aspect, one or more example embodiments relates to a CT device comprising a CT detector module according to one or more example embodiments.

The advantages of the CT device essentially correspond to the advantages of the CT detector module. Features, advantages or alternative embodiments mentioned herein may also be applied to the other claimed subject and vice versa.

The CT device can be an X-ray source, a CT detector module and a supply unit. The X-ray source and the CT detector module can be arranged opposite one another. The X-ray source can be designed to expose the CT detector module to X-ray radiation along an X-ray incidence direction. The CT device can in addition comprise a gantry with a rotor. The X-ray source and the CT detector module can be arranged in a defined arrangement on the rotor, in particular integrated in the rotor or fastened to the rotor. The rotor can be mounted so as to be able to rotate about an axis of rotation. An examination object to be imaged, for example a human and/or animal patient and/or an examination phantom, can be placed on a patient table and moved along the axis of rotation through the gantry.

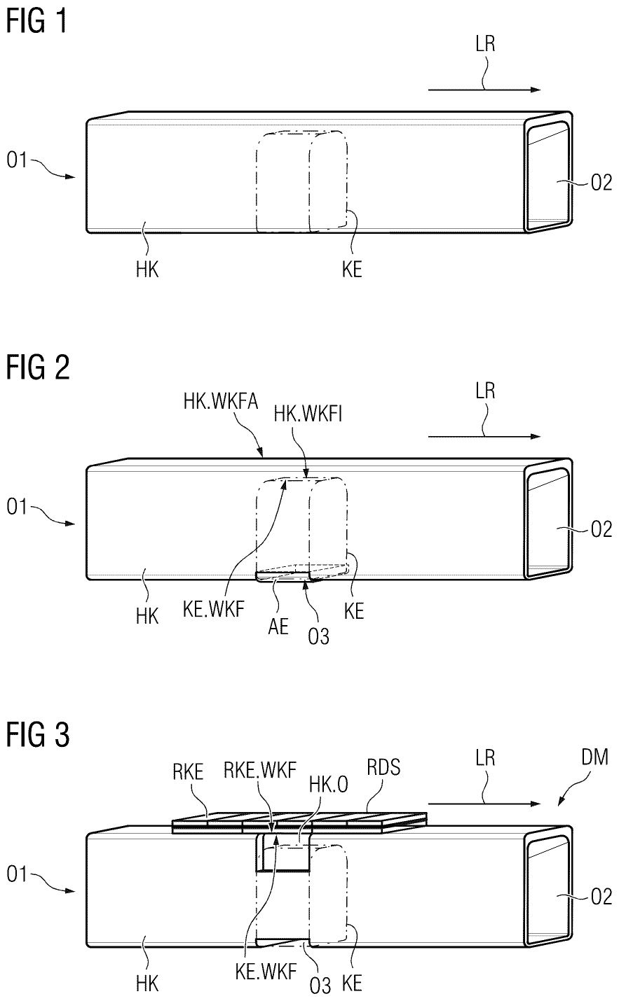

FIG. 1 shows a schematic representation of an advantageous embodiment of a module carrier. The module carrier can have a one-piece and tunnel-shaped hollow body HK. Moreover, the hollow body HK can have an elongated hollow space HR and at least two openings O1 and O2. Two openings O1 and O2 of the at least two openings O1 and O2 can be arranged opposite along a longitudinal direction of extent LR of the hollow body HK. The hollow body HK can be designed to be arranged on its outer side on an X-ray converter element (not shown here). Moreover, the hollow body HK can be designed to receive at least one cooling element KE by insertion by way of at least one of the at least two openings O1, O2 in its hollow space HR. In addition, the hollow body HK can be designed to enable fluid to flow through along its longitudinal direction of extent LR. Moreover, the hollow body HK can have a heat transfer section which is designed to provide a heat-conducting contact between the at least one cooling element KE and the X-ray converter element.

Advantageously, the hollow space HR can run in a straight line along the longitudinal direction of extent LR of the hollow body HK. Moreover, the heat transfer section can have respectively an opening HK.O for each of the cooling elements KE to be received. In this operating state of the module carrier, the at least one cooling element KE when arranged in the hollow space HR can have a direct heat-conducting contact with the X-ray converter element.

The hollow body HK can have at least one first and at least one further section along its longitudinal direction of extent LR. In this case, the at least one first section can be designed to receive the at least one cooling element KE. Furthermore, the at least one second section can have a flow-conducting internal structure.

Advantageously, the hollow body HK can have a closed polygon profile transverse to its longitudinal direction of extent LR.

FIG. 2 shows a schematic representation of a further advantageous embodiment of a module carrier. In this case, the heat transfer section can have respectively a heat contact surface HK.WKFI on the inner side of the hollow body HK for each of the cooling elements KE to be received and on the outer side at least one further heat contact surface HK.WKFA. The heat transfer section can be designed to transfer heat between the heat contact surfaces of the inner side HK.WKFI and outer side HK.WKFA of the hollow body HK. Moreover, in an operating state of the module carrier, the at least one cooling element KE, in particular a heat contact surface of the at least one cooling element KE. WKF, when arranged in the hollow space HR, can have a direct heat-conducting contact with the respective heat contact surface on the inner side HK.WKFI and the X-ray converter element (not shown here), when arranged on the outer side of the hollow body, can have a direct heat-conducting contact with the heat contact surface on the outer side HK.WKFA.

Furthermore, the hollow body HK can have a further opening O3 which is arranged away from its longitudinal direction of extent LR. The hollow body HK can be designed to receive the at least one cooling element KE by insertion by way of the further opening O3 in its hollow space HR. In addition, the module carrier can have a cover element AE. In this case, in an operating state of the module carrier, the at least one cooling element KE can be arranged in the hollow space HR and the cover element AE fastened to the hollow body HK. In addition, the cover element AE can seal the further opening O3 at least partially.

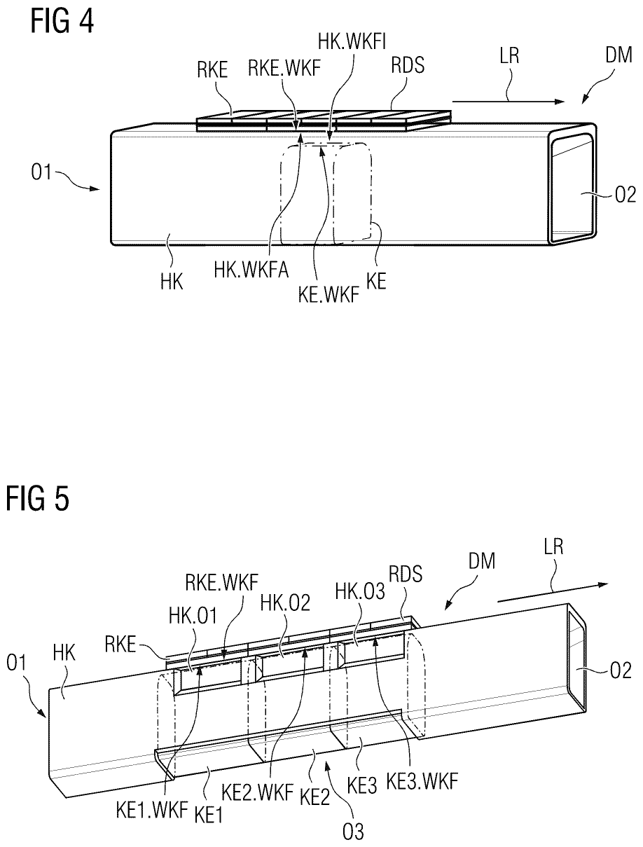

FIG. 3 shows an advantageous embodiment of a CT detector module. The CT detector module can comprise at least one X-ray converter element RKE, a module carrier and at least one cooling element KE. The X-ray converter element RKE can have an upper side and an underside. The X-ray converter element RKE can have on its upper side an X-ray detector layer RDS and on its underside a heat contact surface RKE. WKF. In an operating state of the CT detector module, the at least one cooling element KE can be arranged in the hollow space HR of the hollow body HK. Moreover, in the operating state, the X-ray converter element RKE can be arranged on the outer side of the hollow body. Furthermore, in the operating state, the at least one cooling element KE can be in heat-conducting contact with the heat contact surface of the X-ray converter element RKE. WKF by way of the heat transfer section. Moreover, the heat transfer section can have respectively an opening HK.O for each of the cooling elements KE to be received. In an operating state of the module carrier, the at least one cooling element KE when arranged in the hollow space HR can have a direct heat-conducting contact with the X-ray converter element RKE. In particular, the at least one cooling element can have a heat contact surface KE. WKF which in the operating state directly thermally contacts the heat contact surface of the X-ray converter element RKE. WKF.

Advantageously, the at least one cooling element KE can be designed in the arrangement in the hollow space HR of the hollow body HK to enable a fluid to flow through along the longitudinal direction of extent LR of the hollow body HK. In the operating state of the CT detector module, the fluid flows through the hollow body HK and the at least one cooling element KE arranged therein along the longitudinal direction of extent LR of the hollow body HK.

FIG. 4 shows a further advantageous embodiment of a CT detector module. In this case, the heat transfer section can have on the inner side of the hollow body HK for each of the cooling elements KE to be received respectively a heat contact surface HK.WKFI and on the outer side at least one further heat contact surface HK.WKFA. The heat transfer section can be designed to transfer heat between the heat contact surfaces of the inner side HK.WKFI and outer side HK.WKFA of the hollow body HK. Moreover, in an operating state of the module carrier, the at least one cooling element KE when arranged in the hollow space HR can have a direct heat-conducting contact with the respective heat contact surfaces on the inner side HK.WKF1 and the X-ray converter element RKE when arranged on the outer side of the hollow body can have a direct heat-conducting contact with the heat contact surface on the outer side HK.WKFA. In particular, the heat contact surface of the X-ray converter element RKE. WKF in the operating state can thermally contact the heat contact surface of the hollow body on the outer side HK.WKFA. In addition, the heat contact surface of the at least one cooling element KE. WKF can thermally contact the heat contact surface of the hollow body on the inner side HK.WKFI in the operating state.

FIG. 5 shows a further advantageous embodiment of a CT detector module. In this case, the hollow body HK can be designed to receive multiple cooling elements KE1, KE2 and KE3 by way of at least one of the at least two openings O1, O2 in its hollow space HR. Furthermore, the multiple cooling elements KE1, KE2 and KE3 can be arranged in a row in the hollow space HR along the longitudinal direction of extent LR of the hollow body HK. In particular, the hollow body HK can have respectively a heat transfer section for each of the cooling elements KE1, KE2, KE3. The cooling elements KE1, KE2, KE3 in the row-shaped arrangement can have a different length along the longitudinal direction of extent LR of the hollow body HK. In addition, a length of the heat transfer sections along the longitudinal direction of extent LR of the hollow body HK can be adapted to the length of the cooling elements KE1, KE2, KE3. Advantageously, the heat transfer section can have respectively an opening HK.O1, HK.O2, HK.O3 for each of the cooling elements KE1, KE2, KE3 to be received. In this case, in the operating state, the one cooling element KE1, KE2, KE3 can be in direct heat-conducting contact with the heat contact surface of the X-ray converter element RKE. WKF via respective heat contact surfaces KE1. WKF, KE2. WKF, KE3. WKF and can be fastened to the X-ray converter element RKE via a fastening means. Alternatively or additionally, the cooling elements KE1, KE2, KE3 can be fastened to the hollow body HK via a further fastening means in the operating state.

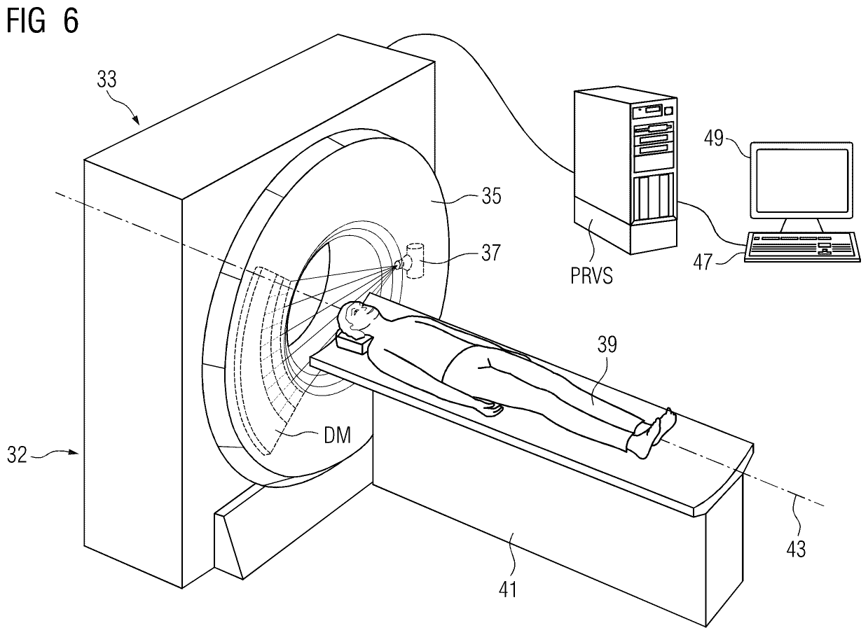

FIG. 6 shows a schematic representation of an advantageous embodiment of a CT device 33, comprising an X-ray source 37, a CT detector module DM and a supply unit PRVS. The X-ray source 37 and the CT detector module DM can be arranged opposite one another. The X-ray source 37 can be designed to expose the CT detector module to X-ray radiation along an X-ray incidence direction. The CT device 33 can comprise a gantry 33 with a rotor 35. The X-ray source 37 and the CT detector module DM can be arranged in a defined arrangement on the rotor 35, in particular integrated in the rotor 35 or fastened to the rotor 35. The rotor 35 can be mounted so as to be able to rotate about an axis of rotation 43. An examination object to be imaged 39 can be placed on a patient table 41 and moved along the axis of rotation 43 through the gantry 33. The supply unit PRVS can be used to control the CT device 32 and to calculate sectional images or volume images of the examination object 39. An input facility 47, for example a keyboard, and an output apparatus 49, for example a screen and/or display, can be connected to the supply unit PRVS, in particular coupled in terms of signal technology. The input facility 47 can be advantageously integrated into the output apparatus 49, for example in the case of an in particular resistive and/or capacitive input display.

The schematic representations shown in the described figures do not depict any scale or size relationships.

Finally, it is pointed out once again that the methods described in detail above and the illustrated apparatuses are merely exemplary embodiments which can be modified by the person skilled in the art in a wide variety of ways without leaving the scope of the invention. Furthermore, the use of the indefinite articles “a” or “one” does not exclude the possibility that the features in question may be present more than once. Likewise, the terms “unit” and “element” do not exclude that the relevant components consist of multiple interacting part components which can where appropriate also be distributed in a spatial manner.

In the context of the present application, the expression “based on” can be understood in particular in the sense of the expression “using”. In particular, a formulation according to which a first feature is generated (alternatively: determined, defined, etc.) based on a second feature does not preclude the first feature being generated (alternatively: determined, defined, etc.) based on a third feature.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially herein accordingly. In relative descriptors used interpreted addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code operations corresponding and thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A module carrier for a CT detector module, comprising:
a one-piece tunnel-shaped hollow body, the hollow body including an elongated hollow space and at least two openings, two openings of the at least two openings are opposite one another along a longitudinal direction of the hollow body, the hollow body configured to,
be arranged on an X-ray converter element on an outer side of the hollow body,
receive at least one cooling element in the hollow space via at least one of the at least two openings, and
enable a fluid to flow through the hollow body along the longitudinal direction of the hollow body,
wherein the hollow body has a heat transfer section configured to provide a heat-conducting contact between the at least one cooling element and the X-ray converter element.

2. The module carrier of claim 1, wherein
the hollow body has a further opening away from the longitudinal direction of the hollow body, and
the hollow body is configured to receive the at least one cooling element in the hollow space via the further opening.

3. The module carrier of claim 2, wherein
the module carrier has a cover element,
in an operating state of the module carrier, the at least one cooling element is in the hollow space and the cover element is fastened to the hollow body, and
the cover element at least partially seals the further opening.

4. The module carrier of claim 1, wherein
the hollow body is configured to receive multiple cooling elements in the hollow space via at least one of the at least two openings, and
the multiple cooling elements are arrangeable along the longitudinal direction of the hollow body in a row in the hollow space.

5. The module carrier of claim 4, wherein
the hollow body has a respective heat transfer section for each of the cooling elements,
the cooling elements in the row have a different length along the longitudinal direction of the hollow body, and
a length of the heat transfer sections along the longitudinal direction of the hollow body corresponding to the length of the cooling elements.

6. The module carrier of claim 4, wherein
the heat transfer section has a respective opening for each of the cooling elements, and
in an operating state of the module carrier, the at least one cooling element has a direct heat-conducting contact with the X-ray converter element when in the hollow space.

7. The module carrier of claim 1, wherein
the heat transfer section has a respective heat contact surface on an inner side of the hollow body for each of the at least one cooling element to be received and has at least one further heat contact surface on the outer side of the hollow body,
the heat transfer section is configured to transfer heat between the heat contact surfaces of the inner side of the hollow body and the outer side of the hollow body, and
in an operating state of the module carrier,
the at least one cooling element, when arranged in the hollow space, has a direct heat-conducting contact with the respective heat contact surface on the inner side of the hollow body, and
the X-ray converter element, when arranged on the outer side of the hollow body, has a direct heat-conducting contact with the heat contact surface on the outer side of the hollow body.

8. The module carrier of claim 1, wherein
the hollow body has at least one first section and at least one further section along the longitudinal direction of the hollow body,
the at least one first section is configured to receive the at least one cooling element, and
the at least one further section has a flow-conducting internal structure.

9. The module carrier of claim 1, wherein the hollow body has a polygon profile or an oval profile transverse to the longitudinal direction of the hollow body.

10. A CT detector module comprising:
at least one X-ray converter element;
the module carrier of claim 1; and
the at least one cooling element,
wherein the X-ray converter element has an upper side and an underside,
the X-ray converter element has an X-ray detector layer on the upper side and has a heat contact surface on the underside, and
in an operating state of the CT detector module,
the at least one cooling element is in the hollow space of the hollow body,
the X-ray converter element is on the outer side of the hollow body, and
the at least one cooling element is in heat-conducting contact with the heat contact surface of the X-ray converter element via the heat transfer section.

11. The CT detector module of claim 10, wherein
the at least one cooling element in the hollow space of the hollow body is configured to enable the fluid to flow through along the longitudinal direction of the hollow body, and
in the operating state of the CT detector module, the fluid flows through the hollow body and the at least one cooling element along the longitudinal direction of the hollow body.

12. The CT detector module of claim 10, further comprising:
multiple cooling elements arranged along the longitudinal direction of the hollow body in a row in the hollow space.

13. The CT detector module of claim 10, wherein the heat transfer section has a respective opening for each of the at least one cooling element, and in the operating state,
the at least one cooling element is in direct heat-conducting contact with the heat contact surface of the X-ray converter element, and
the at least one cooling element is fastened to the X-ray converter element via a fastening means.

14. The CT detector module of claim 10, wherein in the operating state the at least one cooling element is fastened to the hollow body via a further fastening means.

15. A CT device, comprising the CT detector module of claim 10.

16. The module carrier of claim 3, wherein
the hollow body is configured to receive multiple cooling elements in the hollow space via at least one of the at least two openings, and
the multiple cooling elements are arrangeable along the longitudinal direction of the hollow body in a row in the hollow space.

17. The module carrier of claim 16, wherein
the heat transfer section has a respective opening for each of the cooling elements, and
in an operating state of the module carrier, the at least one cooling element has a direct heat-conducting contact with the X-ray converter element when in the hollow space.

18. The module carrier of claim 16, wherein
the heat transfer section has a respective heat contact surface on an inner side of the hollow body for each of the at least one cooling element to be received and has at least one further heat contact surface on the outer side of the hollow body,
the heat transfer section is configured to transfer heat between the heat contact surfaces of the inner side of the hollow body and the outer side of the hollow body, and
in an operating state of the module carrier,
the at least one cooling element, when arranged in the hollow space, has a direct heat-conducting contact with the respective heat contact surface on the inner side of the hollow body, and
the X-ray converter element, when arranged on the outer side of the hollow body, has a direct heat-conducting contact with the heat contact surface on the outer side of the hollow body.

19. The module carrier of claim 16, wherein
the hollow body has at least one first section and at least one further section along the longitudinal direction of the hollow body,
the at least one first section is configured to receive the at least one cooling element, and
the at least one further section has a flow-conducting internal structure.

20. The module carrier of claim 16, wherein the hollow body has a polygon profile or an oval profile transverse to the longitudinal direction of the hollow body.
